(19) United States
(12) Patent Application Publication
(10) Pub. No.: US 2023/0355713 A1
(43) Pub. Date: Nov. 9, 2023

(54) PEPTIDE FOR THERAPEUTIC APPLICATIONS IN THE DERMATOLOGICAL FIELD

(71) Applicant: Dompe' Farmaceutici S.P.A., Milano (IT)

(72) Inventor: Pablo PELLEGRINI, Milano (IT)

(21) Appl. No.: 18/021,979

(22) PCT Filed: Aug. 19, 2021
(86) PCT No.: PCT/IB2021/057631

§ 371 Date: Feb. 17, 2023

(30) Foreign Application Priority Data
Aug. 20, 2020 (IT) 102020000020287

(51) Int. Cl. A61K 38/18; A61P 17/00; A61K 45/06

(52) U.S. Cl. CPC A61K 38/185; A61P 17/00; A61K 45/06

(57) ABSTRACT

The present invention relates to a peptide of 14 amino acids having the sequence ID No. 1 or peptide of up to 16 amino acids having a sequence with at least 85%, preferably at least 90%, and preferably at least 95% identity with sequence ID No. 1, or a derivative and/or salt thereof, capable of promoting skin pigmentation and/or innervation in skin dyschromia and/or de-innervation diseases. A pharmaceutical composition comprising such peptide and at least one pharmaceutically acceptable excipient are a further object of the invention.

### FIELD OF THE INVENTION

The present invention relates to a peptide capable of promoting skin pigmentation and/or innervation in skin dyschromia and/or de-innervation diseases. A pharmaceutical composition comprising such peptide and at least one pharmaceutically acceptable excipient are a further object of the invention.

### PRIOR ART

Nerve growth factor (NGF) is a neurotrophic and neuropeptide factor primarily involved in regulating the growth, maintenance, proliferation and survival of certain target neurons.

NGF was discovered by Prof. Rita Levi-Montalcini, at the Zoology Institute of the Washington University of St. Louis (Levi-Montalcini R., Harvey Lect., 60:217, 1966), and her discovery represented a notable progress in the study of the mechanisms of growth and differentiation of the nerve cell, as NGF is able to influence the development and conservation of the biological functions of neurons and their regeneration.

Numerous in vitro and in vivo studies have demonstrated the pathophysiological importance of NGF in the prevention of neuronal damage of a surgical, chemical, mechanical and ischemic nature, making it the ideal candidate for the treatment of various conditions of the central and peripheral nervous system (Hefti F., J. Neurobiol., 25: 1418, 1994; Fricker J., Lancet, 349:480, 1997). In fact, for many years clinical studies have been carried out on patients suffering from Parkinson's disease and Alzheimer's disease, by intracerebral administration of murine NGF (see, for example, Olson L. et al., J. Neural Transm, (Parkinson's Disease and Dementia Section), 4: 79-95, 1992). The results of these studies confirmed the observations made in animal models and showed the absence of possible side effects following the administration of murine NGF. This feature was later confirmed for human recombinant NGF (Petty B G et al., Annals of Neurology, 36:244-246, 1994).

Immune, neuronal and skin cells play a key role in the onset of various dermatological pathologies associated with dyschromia, such as psoriasis, atopic dermatitis, vitiligo, herpes.

NGF receptors are expressed on sensory nerves, keratinocytes, melanocytes, fibroblasts, hair follicles and various immune cells, playing an important role in skin homeostasis.

Keratinocytes are a key cellular component of both homeostasis and pathophysiological processes in the skin and have been shown to produce a number of cytokines and to be stimulated by various growth factors. NGF mRNA has been shown in vitro to be maximal when keratinocytes are in exponential growth phase (Growth-regulated synthesis and secretion of biologically active nerve growth factor by human keratinocytes. J Biol Chem 1991; 266: 21718-21722).

In keratinocytes of various species, NGF regulates proliferation and differentiation, and protects those of human nature from apoptosis by activating the high affinity receptor (trkA), showing a known anti-aging and skin regeneration activity (Nerve growth factor: its significance in cutaneous biology. J Invest Dermatol Symp Proc 2: 31-36, 1997), and for maintaining the integrity of the epidermis.

Indeed, Trka plays a key role in activating the signal transduction pathways that promote growth and survival, and which are also involved in skin innervation processes.

In melanocytes, survival and dendrite formation is increased by NGF after UV irradiation (The trk family of receptors mediates nerve growth factor and neurotrophin-3 effects in melanocytes J. Clin. Invest. 94: 1550-1562, 1994).

The prior art shows how the regulation of NGF can favor the treatment of both skin diseases associated with dyschromic phenomena, and diseases associated with de-innervation, such as viral rashes, such as herpes, and rare syndromes such as trigeminal trophic, which often occurs in areas previously affected by herpes zooster.

The treatment of vitiligo is currently entrusted to therapies that have significant side effects (Am. J. Clin. Dermatol., 2017, 18:733-744). In particular, treatment with corticosteroids, administered both topically and orally, cannot be prolonged beyond 3 weeks. Even the treatment of choice represented by narrow-band phototherapy (NB-UVB phototherapy), despite the average quality of the results obtained (re-pigmentation greater than 75% in 63% of cases) can cause acute undesirable effects such as itching, erythema, burns, xerosis.

The international publication WO2013065078A2 revealed that the topical administration on the skin of preparations containing NGF is effective in obtaining an intensification of the skin color, i.e. an increase in pigmentation, in the case of healthy skin, not affected by dyschromatosis, as well as an improvement in the dermatological conditions involving skin achromia or hypochromia, as in the case of vitiligo.

Despite research developments, there is still a problem of improving the therapy of skin discolorations and skin de-innervation diseases.

### SUMMARY OF THE INVENTION

The Applicant has faced the problem of improving the therapy of skin dyschromias and skin de-innervation diseases. In particular, the Applicant has focused its research in the field of peptides derived from NGF, finding that the NGF 1-14 peptide having the sequence ID No. 1 is particularly effective in activating the production of melanin by melanocytes.

As will be demonstrated in Example 1 of the experimental part, the NGF 1-14 peptide tested in vitro on primary cell cultures of normal human melanocytes results in a comparable increase in the melanin content per cell compared to the whole NGF protein, albeit at higher concentrations (2-3 log).

Surprisingly with respect to the activity demonstrated in vitro, as will be demonstrated in Example 2a, the NGF 1-14 peptide tested in vivo on a mouse model of human vitiligo shows a more uniform and homogeneous repigmentation than the whole protein, at the same dose; as shown in Example 2b, in vivo the NGF 1-14 peptide surprisingly determines a greater increase in the density of the nerve endings, at the same dose.

As will be shown in Example 3, the NGF 1-14 peptide administered by intraplantar route to Sprague-Dawley rats shows much higher tolerability even at the maximum dose, compared to NGF. The result of this test is very important in light of the fact that the pain caused at the site of administration by NGF and consequently the poor skin tolerability of the whole protein administered intradermally or intramuscularly is known. This effect has been extensively studied and has been shown to be intrinsically linked to the mechanism of action, in particular to the activation of NGF receptors. For this reason, the observed tolerability of the NGF 1-14 peptide administered intradermally is surprising.

A further advantage of the NGF1-14 peptide with respect to the whole protein is represented by the fact that it can be easily synthesized with conventional peptide synthesis approaches and stored under mild conditions and for prolonged periods, with a consequent significant reduction in production costs.

The Applicant also noted that some changes in the number and type of amino acids of the NGF1-14 peptide can be made without substantially altering the activity thereof.

Therefore, a first object of the present invention relates to a peptide of 14 amino acids having the sequence ID No. 1 or a peptide of up to 16 amino acids having a sequence with at least 85%, preferably at least 90%, and preferably at least 95% identity with sequence ID No. 1 or a derivative and/or salt thereof, for use in the treatment and/or prevention of skin dyschromia and/or de-innervation diseases of the skin.

A second object of the present invention relates to a pharmaceutical composition consisting of a peptide of 14 amino acids having the sequence ID No. 1 or a peptide of up to 16 amino acids having a sequence with at least 85%, preferably at least 90%, and preferably at least 95% identity with sequence ID No. 1 or a derivative and/or salt thereof, and at least one pharmaceutically acceptable excipient, for use in the treatment and/or prevention of skin dyschromia and/or de-innervation diseases of the skin.

A third object of the present invention relates to a therapeutic method for the treatment and/or prevention of skin dyschromia and/or skin de-innervation diseases in a person in need thereof, comprising the topical application of an effective amount of a composition consisting of a peptide of 14 amino acids having the sequence ID No. 1 or a peptide of up to 16 amino acids having a sequence with at least 85%, preferably at least 90%, and preferably at least 95% identity with sequence ID No. 1, or a derivative and/or salt thereof, and at least one pharmaceutically acceptable excipient.

By the term “effective amount” used herein it is meant an amount of peptide or composition comprising it sufficient to induce a significant benefit, including, independently or in combination, the benefits disclosed herein, but low enough to avoid serious side effects, i.e. to provide a reasonable benefit/risk ratio, in the context of the judgment of a man skilled in the art. The effective amount can vary within a wide range depending on known factors, for example the type of pathology, the severity of the disease, the patient's body weight, the dosage form, the number of administrations per day. However, the man skilled in the art can determine the optimum quantity in a simple and ordinary way.

### BRIEF DESCRIPTION OF THE FIGURES

FIG. 1 shows the paw retraction latency of animals treated with saline (black circle, solid line), with NGF 1-14 10 μg/50 μL (white square), NGF 1-14 20 μg/50 μL (black circle—broken line) and rh-NGF 10 μg/50 μL mL (black triangle) at 1, 3, 5 and 24 hours after injection.

### DETAILED DESCRIPTION OF THE INVENTION

Preferably, in an embodiment according to the first object of the present invention, the peptide has a length of 14 amino acids and sequence ID No. 1 or a derivative and/or salt thereof.

The sequence ID No. 1 according to the present invention is represented by the sequence SSSHPIFHRGEFSV.

The abbreviations of the amino acid sequences used herein comply with the IUPAC-IUB nomenclature shown in the following Table A.

TABLE A

| | | | | | |
|---|---|---|---|---|---|
| Alanine | Ala | A | Arginine | Arg | R |
| Asparagine | Asn | N | Aspartic acid | Asp | D |
| Cysteine | Cys | C | Glutamic acid | Glu | E |
| Glutamine | Gln | Q | Glycine | Gly | G |
| Histidine | His | H | Isoleucine | lle | I |
| Leucine | Leu | L | Lysine | Lys | K |
| Methionine | Met | M | Phenylalanine | Phe | F |
| Proline | Pro | P | Serine | Ser | S |
| Threonine | Thr | T | Tryptophan | Trp | W |
| Tyrosine | Tyr | Y | Valine | Val | V |

The peptides according to the invention can have at least 85%, at least 90% and at least 95% identity with the sequence ID No. 1 when they are optimally aligned.

Optimal sequence alignment can be conducted with various known methods and with the computer implementation of known algorithms (e.g. BLAST, TFASTA, BESTFIT, as in Wisconsin's Genetics Software Package, Release 7.0, Genetics Computer Group, Madison, WI). The BLAST algorithm can also be used (Altschul et al., Mol. Biol. (1990), 215, 403-410) whose software can be obtained from the National Center for Biotechnology Information www.ncbi.nlm.nih.gov/.

By “percentage sequence identity” with respect to a peptide sequence it is meant the percentage of identical residues in two sequences. The percentage sequence identity (% SI) is calculated with the following formula:

% SI=(nt−nd)×100/nt

where nt is the number of residues in the base sequence and nd is the total number of non-identical residues in the compared sequence when aligned so that the maximum number of amino acids is identical. Consequently, an SSSHPIFHRGDFDFSV sequence will have a sequence identity of approximately 92.8% with sequence ID No. 1 (nd=1 and nt=14).

Preferably, in another embodiment according to the first object of the present invention, the peptide has a length of up to 16 amino acids and a sequence with at least 85%, preferably at least 90%, and preferably at least 95% identity with the sequence ID No. 1, or a derivative and/or salt thereof; more preferably, according to such embodiment, said peptide has a length of up to 15 amino acids, more preferably of 14 amino acids.

The variation of the amino acid sequence in the peptides comprising the ID No. 1 of the present invention comprises the conservative substitution of amino acids which do not affect peptide activity. Substitutions capable of maintaining peptide activity are selected based on (a) effectiveness in maintaining the backbone structure of the peptide in the substitution area, such as three-dimensional foil or helical structures, (b) effectiveness in maintaining the electric charge or the hydrophobicity of the molecule in the target area, or (c) effectiveness in maintaining the bulk of the side chain.

Examples of conservative substitution belong to the group of basic amino acids (arginine, lysine and histidine), acid amino acids (glutamic and aspartic acids), polar amino acids (glutamine and asparagine), hydrophobic amino acids (leucine, isoleucine, valine and methionine), aromatic amino acids (phenylalanine, tryptophan and tyrosine) and small amino acids (glycine, alanine, serine and threonine).

Substitutions of amino acids which generally do not alter the specific activity are known in the art of the present invention.

The most common alterations are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly, and the opposite alterations. Another example of conservative substitution is shown in the following Table C.

TABLE C

| Starting | Possible | Preferred |
|---|---|---|
| amino acid | substitution | substitution |
| Ala (A) | V; L; I | V |
| Arg (R) | K; Q; N | K |
| Asn (N) | Q; H; D, K; R | Q |
| Asp (D) | E; N | E |
| Cys (C) | S; A | S |
| Gln (Q) | N; E | N |
| Glu (E) | D; Q | D |
| Gly (G) | A | A |
| His (H) | N; Q; K; R | R |
| Ile (I) | L; V; M; A; F; | L |
| Leu (L) | I; V; M; A; F | I |
| Lys (K) | R; Q; N | R |
| Met (M) | L; F; I | L |
| Phe (F) | L; V; 1; A; Y | Y |
| Pro (P) | A | A |
| Ser (S) | T | T |
| Thr (Thr) | S | S |
| Trp (W) | Y; F | Y |
| Tyr (Y) | Trp; F; T; S | F |
| Val (V) | I; L; M; F; A; | L |

Preferably according to the first object of the present invention, said derivative comprises an N and/or a C terminal of said peptide which is chemically modified or protected with an organic compound; more preferably, said organic compound is selected from the group consisting of phosphoryl, glycosyl, acyl, alkyl, carboxyl, hydroxyl, biotinyl, ubiquitinyl, and amido groups.

Depending on its length, the peptide of the present invention can be synthesized by a method well known in the art, for example, by an automatic peptide synthesizer, or produced by a genetic engineering technology. For example, a fusion gene encoding a fusion protein which includes a fusion partner and the peptide of the present invention is prepared by genetic engineering and then transformed into a host cell to express the fusion protein. Next, the peptide of the present invention is cleaved and isolated from the fusion protein using a protease or a compound so as to produce the desired peptide. To this end, between the polynucleotides encoding the fusion partner and the peptide of the present invention, a DNA sequence can be inserted which encodes the amino acid residues which can be cleaved by a protease such as the factor Xa or enterokinase, or a compound such as CNBr or hydroxylamine.

Preferably, in an embodiment according to the second object of the present invention, the pharmaceutical composition comprises from 10 to 10,000 μg/mL, preferably from 100 to 5,000 μg/mL, and more preferably from 200 to 4,000 μg/mL of a peptide having a length of 14 amino acids and sequence ID No. 1 or a derivative and/or salt thereof.

Preferably, in another embodiment according to the second object of the present invention, the pharmaceutical composition comprises from 10 to 10,000 μg/mL, preferably from 100 to 5,000 μg/mL, and more preferably from 200 to 4,000 μg/mL of a peptide having a length of up to 16 amino acids and a sequence with at least 85%, preferably at least 90%, and preferably at least 95% identity with sequence ID No. 1 or a derivative and/or salt thereof.

According to the second object of the present invention, the pharmaceutical composition can contain a variety of other optional components suitable for making such compositions more pharmaceutically acceptable or for providing them with further benefits of use. Such conventional optional ingredients are well known to those skilled in the art. These include all pharmaceutically acceptable ingredients such as those described for example in Rowe et al.; “Handbook of Pharmaceutical Excipients”, 2009, Pharmaceutical Press.

The type of vehicle or excipient used in the present invention depends on the type of product desired. The compositions useful in the present invention can be a large variety of product forms. These include, but are not limited to, lotions, creams, gels, sticks, sprays, ointments, pastes, mousses.

These product forms may include several types of vehicles, including, but not limited to, solutions, aerosols, emulsions (including oil-in-water and water-in-oil), gels, solid compositions and liposomes.

The compositions of the present invention can comprise water, in different amounts depending on the form of the composition. The amount of water, if present, can range from less than 1% to more than 99% by weight with respect to the weight of the total composition. The aqueous compositions of the present invention are particularly formulated as aqueous lotions or as aqueous emulsions or as water-in-oil or oil-in-water emulsions or as multiple emulsions (triple oil-in-water-in-oil or water-in -oil-in-water emulsion).

The solid compositions, spray compositions and water-in-oil creams generally consist of quantities of water lower than 10%, preferably lower than 5% by the total weight of the composition. The roll-on compositions, aqueous compositions and deodorants generally consist of an amount of water ranging from about 15% to about 99%, preferably from about 30% to about 90%, even more preferably from about 50% to about 80%, by weight with respect to the total weight of the composition.

The compositions of the present invention can comprise one or more volatile solvents. If present, the volatile solvent or solvent mixture will generally be at a level between about 10% and about 90%, preferably between about 25% and about 75%, even more preferably between about 35% and about 65%, by weight with respect to the total weight of the composition. The solvents used herein are preferably volatile organic solvents.

As used herein, the term “volatile” refers to substances with a significant amount of vapor pressure under ambient conditions, as understood by those skilled in the art.

The volatile solvents to be used herein will preferably have a vapor pressure of about 2 kPa or more, preferably about 6 kPa or more, at 25° C. The volatile solvents to be used will preferably have a boiling point in a normal atmosphere (1 atm) lower than about 150° C., more preferably lower than about 100° C., even more preferably lower than about 90° C., and even more preferably lower than about 80° C.

Preferably, the volatile solvents to be used herein will be relatively odorless and safe for use on human skin. Suitable volatile solvents include, but are not limited to C₁-C₄ alcohols, volatile silicones and mixtures thereof. The preferred volatile solvents are C₁-C₄ alcohols and mixtures thereof. Ethanol is the preferred solvent for the present use.

The compositions of the present invention can also comprise one or more non-volatile solvents. If present, the non-volatile solvent or solvent mixture will generally be at a level between about 1% and about 20%, preferably about 2% to about 10%, even more preferably about 3% to about 5%, by weight with respect to the total weight of the composition. Suitable non-volatile solvents include, but are not limited to, benzyl benzoate, cetearyl alcohol, cetyl alcohol, diethyl phthalate, isopropyl myristate, dimethicone, caprylylmethicone and mixtures thereof.

Several other additional ingredients may be present in the compositions of the present invention. These include, but are not limited to, hydrophilic polymers selected from polyethylene glycols (PEG), polyvinylpyrrolidones (PVP), hydroxypropylmethylcellulose (HPMC) and poloxamers; UV stabilizers such as benzophenone-3; antioxidants such as tocopheryl acetate; preservatives such as phenoxyethanol, benzyl alcohol, methylparaben, propylparaben; pH adjusting agents such as lactic acid, citric acid, sodium citrate, succinic acid, phosphoric acid, sodium hydroxide, sodium carbonate; deodorants and antimicrobials, such as farnesol, zinc phenolsulfonate and ethylhexylglycerin; humectants such as tribehenin, glycerin; skin conditioning agents such as allantoin; cooling agents such as trimethyl isopropyl butanamide and menthol; hair treatment ingredients such as panthenol, pantethine, pantothine, panthenyl ethyl ether and their combinations; propellants such as propane, isopropane, butane and isobutene; salts in general, such as potassium acetate and sodium chloride and mixtures thereof; perfumes and dyes.

If present, these additional ingredients will preferably be present at a level of less than 10%, preferably less than 5% by weight of the total weight of the composition.

Preferably, in an embodiment according to the second object of the present invention, the pharmaceutical composition is for topical administration, more preferably in the form of lotion, cream, gel, stick, spray, ointment, paste, mousse; even more preferably it is in the form of a cream.

According to the first, second or third object of the present invention, the treatment and/or prevention of skin dyschromia includes hypopigmentation and/or depigmentation disorders of the hypopigmentary skin, disorders of the melanic pigmentation of the skin, such as specific pathologies of post-trophic, neurotrophic, post-traumatic, post-infectious, post-surgical, autoimmune, genetic, metabolic, nutritional, endocrine, chemical, physical, dystrophic, degenerative or post-inflammatory origin.

Preferably, according to the first, second or third object of the present invention, the treatment and/or prevention of skin dyschromia includes: vitiligo, bilateral vitiligo, acrofacial vitiligo, generalized vitiligo, focal vitiligo, segmental vitiligo, universal vitiligo, perinevic or Sutton's nevus vitiligo, leukoderma, skin dyschromia, piebaldism, pityriasis alba, pityriasis versicolor, idiopathic and post-inflammatory guttata hypomelanosis, acromic or depigmented nevi, progressive macular hypomelanosis, hypomelanosis caused by metabolic or nutritional or endocrine disorders, hypomelanosis caused by chemical, physical or pharmaceutical agents, infectious and post-infectious hypomelanosis, inflammatory edipomelanosis.

In an embodiment according to the first or second object of the invention relating to the peptide or composition for use in the treatment and/or prevention of skin dyschromia, said peptide or said composition is in combination with a steroid-based compound or composition, or based on calcitriol.

In another embodiment according to the first or second object of the invention, relating to the peptide or composition for use in the treatment and/or prevention of skin de-innervation diseases, said peptide or said composition is in combination with an antiviral compound or composition.

Preferably, according to the third object of the present invention, the method of treatment and/or prevention of skin dyschromia comprises the oral, injectable or topical, simultaneous or sequential administration of a steroid-based or calcitriol-based compound or composition (activated vitamin D).

According to the first, second or third object of the present invention, the treatment and/or prevention of skin de-innervation diseases includes skin de-innervation disorders of viral and/or inflammatory origin.

Preferably, according to the first, second or third object of the present invention, the treatment and/or prevention of skin de-innervation diseases includes trigeminal trophic syndrome, post-herpes zoster itching, scalp dysesthesia, paresthetic notalgia, pruritus brachioradialis, meralgia paresthetica, itching associated with keloids or burn scars, small fiber neuropathy in various systemic diseases, all of which are difficult to treat or lack effective treatment.

Preferably, according to the third object of the present invention, the method of treatment and/or prevention of skin de-innervation diseases of viral origin comprises the oral, injectable or topical, simultaneous or sequential administration of an antiviral compound or composition.

Preferably, according to the third object of the present invention, the method of treatment and/or prevention of skin dyschromia and/or skin de-innervation diseases does not require a photo-activation treatment, favoring adherence to the treatment by the patient.

The invention will be further described in the following examples, which do not limit the scope of the invention described in the claims.

### EXAMPLE 1

### In Vitro Activity

The in vitro ability of the NGF 1-14 peptide and the NGF whole protein to activate melanin production by melanocytes was compared.

According to already established protocols, primary cell cultures of normal human melanocytes were seeded in Petri dishes and cultured for one week, with and without the presence of the NGF 1-14 peptide at various concentrations, using the whole human NGF protein as a control. The melanin content was measured with the ELISA enzyme immunoassay. The maximum melanin concentration, measured as the difference from baseline, was experimentally determined from the plateau of the dose response curves of NGF and NGF 1-14. The EC50 represents the concentration of NGF 1-14 or NGF at which 50% of the maximum concentration of cellular melanin is observed. The experimental data are shown in Table 1.

TABLE 1

| | NGF 1-14 | NGF |
|---|---|---|
| EC50 | 1.2 μM | 60 pM |

As reported in Table 1, the EC50 of NGF 1-14 is 1.2 μM, while NGF's EC50 is 60pM. These data show that NGF 1-14 is able to determine an increase in the melanin content per cell comparable to the whole protein NGF, even if at concentrations higher than 2-3 log.

On the other hand, proliferation and diffusion of melanocytes did not show significant differences.

### EXAMPLE 2

### 2a—In Vivo Activity—Efficacy Test

The in vivo capacity of skin re-pigmentation by the NGF 1-14 peptide and the whole NGF protein was compared.

The study was conducted on a mouse model of human vitiligo.

SCF transgenic mice of the C57BL6 line, treated for 10-12 weeks to induce vitiligo using CD8+T cells specific for melanocytes, were shaved twice a week in the dorsal part affected by depigmentation, with an electric razor, in an area of about 6 cm×6 cm. The animals were divided into two groups of equal numbers (8 animals per group). On a first group of animals, topical administration was carried out in two sites of the shaved area spaced from both sides of the midline, applying on the left a topical preparation containing 200 μg/mL of NGF peptide in a base cream vehicle, while on the right side only the base cream (control) was applied. The same procedure was repeated on the second group of animals, applying on the left a topical preparation containing 200 μg/mL of NGF 1-14 peptide in a base cream vehicle, while on the right side only the base cream (control) was applied.

The application was spread evenly twice a day for 8 weeks, in the central part of the shaved area, on a surface of about 2 cm×2 cm. Starting from the second day and thereafter, the areas of interest were cleaned with a cloth before applying the preparations. The animals were kept away from possible sources of ultraviolet light irradiation.

Quantification of epidermal depigmentation was performed using a subjective scoring system based on a 0 to 5 point scale, where 5 represents the most severe degree of depigmentation, reported in the literature (Rebecca L. et al., Curr Protoc Immunol. (2019), 124(1)—February). A trained researcher blinded to the details of the experiment performed the evaluation at the treated site.

At the end of the study, 80% of the control animals treated with base cream showed a severe degree of depigmentation with a score greater than or equal to 4 both in the group of animals treated with NGF and in the group treated with NGF 1-14 for which the group of control was considered unique. Using the Assessment System described in the publication cited above, a clear effect on pigmentation was observed both in the group of animals treated with NGF 200 μg/mL in base cream and in the group treated with NGF 1-14 200 μg/mL in base cream.

In the first group of animals treated with NGF, 37.5% of the animals showed a degree of depigmentation less than or equal to 2, 37.5% showed a moderate degree equal to 3 and 25% a severe degree greater than or equal to 4.

In the second group of animals treated with NGF 1-14 at the same concentration 62.5% of the animals showed a degree of depigmentation less than or equal to 2, 25% showed a moderate degree equal to 3 only 12.5% a degree severe greater than or equal 4.

As shown by the above data, the complexion of the areas treated with the NGF 1-14 peptide showed a substantial reduction in depigmentation, compared with the complexion of the area treated with the vehicle alone.

In the comparison between peptide NGF 1-14 and NGF whole protein, the repigmentation was substantially comparable, but the percentage of animals with a degree of depigmentation less than or equal to 2 was found to be 62.5% in the group treated with NGF 1-14 compared 37.5% in the NGF-treated group.

The percentage of animals with a degree of depigmentation greater than or equal to 4 was also reduced by 50% in the group treated with NGF 1-14 compared to the group treated with whole protein.

Surprisingly with respect to in vitro activity, these data show that the NGF 1-14 peptide determines a more uniform and homogeneous repigmentation than the whole protein, at the same dose.

### 2b—Histological Test

The in vivo ability of the NGF 1-14 peptide and the NGF whole protein to increase the density of nerve endings was compared.

After the treatment described in example 2a, the animals were sacrificed and skin samples (3 biopsies per point) were taken both from the area treated with NGF1-14 or with NGF, and from the untreated area, for each animal, for the purpose of histological examination.

To evaluate the density of sensory nerve fibers, antibodies directed against the neurofilament protein 200 (NF200; Sigma) specific for large diameter fibers, and antibodies directed against the transient receptor potential vanilloid 1 (TRPV1, Neuromics) were used to measure peripheral sensory fibers (small diameter).

The experimental data are shown in Table 2.

TABLE 2

| | Group 1 | | Group 2 | |
|---|---|---|---|---|
| | | NGF 1-14 | | NGF |
| | Base | 200 μg/mL in | Base | 200 μg/mL in |
| Parameters | cream | base cream | cream | base cream |
| Nerve endings | 150 + 72* | 380 + 61 | 150 + 72* | 350 + 50 |
| density (mm/mm³) | | | | |
| NF200 | | | | |
| Nerve termination | 120 + 20* | 340 + 20 | 150 + 72* | 200 + 36 |
| density unit of | | | | |
| measurement | | | | |
| (mm/mm³) TRPV1 | | | | |

*the two groups were considered homogeneous with respect to the control

The results of the histological analysis show a higher density of small and large diameter nerve fibers both in the group treated with NGF and in the group treated with NGF 1-14. As far as the small diameter fibers are concerned, there is a clear advantage of the treatment with NGF 1-14 compared to the treatment with the whole protein NGF.

Without wishing to bind to any interpretative theory, the Applicant believes that topical application on depigmented skin increases innervation in the treated area, exerting a neurotrophic action on de-pigmented or hypo-pigmented tissue. In turn, the increased innervation stimulates the cutaneous melanocytes and allows to obtain a repigmentation of the treated area.

### EXAMPLE 3

### In Vivo Activity—Tolerability Test

The in vivo intra-implant tolerability of the NGF 1-14 peptide was evaluated in comparison with the whole protein NGF.

Tyrode's isotonic solution containing the rh-NGF protein in concentrations from 5 to 20 μg/mL and the vehicle was administered intra-implantally in the dorsal region of Sprague-Dawley rats (10 animals for each concentration), in a volume of 0.05 mL using a very fine syringe.

Tyrode's isotonic solution containing the murine-NGF protein in concentrations from 5 to 20 μg/mL and the vehicle was administered intra-implantally in the dorsal region of Sprague-Dawley rats (10 animals for each concentration), in a volume of 0.05 mL using a very fine syringe.

Tyrode's isotonic solution containing the NGF 1-14 peptide in concentrations from 5 to 20 μg/mL and the vehicle was administered intra-implantally in the dorsal region of Sprague-Dawley rats (10 animals for each concentration), in a volume of 0.05 mL using a very fine syringe.

The solution was administered intra-implantally in the dorsal region of Sprague-Dawley rats (10 animals for each concentration), in a volume of 0.05 mL using a very fine syringe.

After the intra-implant injections, the Plantar thermal hyperalgesia test was conducted. In particular: after 1, 3, 5 and 24 h the thermal sensitivity was determined using the Hargreaves apparatus (HA).

Hypersensitivity to heat was assessed using the rat plantar test apparatus (Ugo Basile, Italy) following the method described by Hargreaves et al. (1988). The plantar test uses three perspex boxes (22×19×25 cm) placed on an elevated glass table. The rats were housed in each box, so that 3 rats could be tested simultaneously in a single apparatus and allowed to acclimate for at least 10 minutes. A mobile infrared heat source was applied to the plantar surface of the hind legs.

Paw retraction latency was defined as the time (in seconds) it took the rat to remove the hind leg from the heat source. The heat source was calibrated to an intensity of 15 IR and a cut-off of 50 sec was applied to prevent tissue damage. The test was performed at 1, 3, 5 and 24 hours after the injection. Each paw was tested twice per session (raw data represents the average of 2 measurements performed at an interval of approximately 5 minutes).

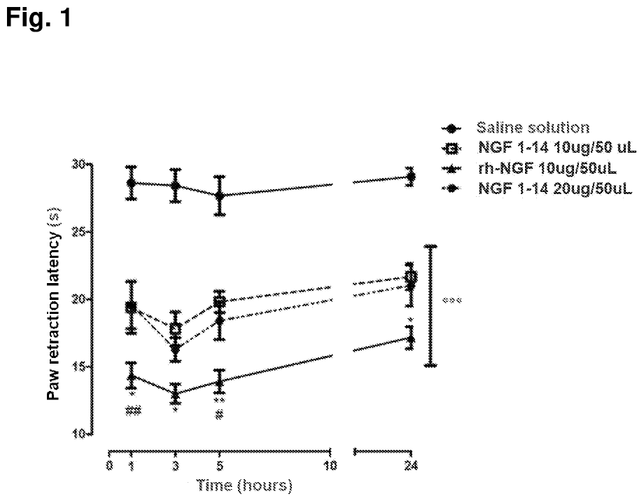

As shown in the graph shown in FIG. 1, in the plantar test, the retraction latency of the paw of the animals treated with vehicle (saline) is unchanged for each time of the study (black circle—solid line). Rats treated with NGF 1-14 10 μg/50 μL (white square), NGF 1-14 20 μ g/50 μ L (black circle—broken line) and rh-NGF 10 μ g/50 μ L mL (black triangle) showed hyperalgesic activity compared to the saline group at all time points of the study (P<0.001). In particular, rh-NGF 10 μg/50 μL showed a more pronounced significant hyperalgesic effect than NGF 1-14 at 10 and 20 μ g/50 μ L (*P<0.05 at 1-3-24 h and **P<0.01 at 5 h).

The test results showed a high tolerability of NGF 1-14, even at the maximum dose of 20 μg/50 μL, much higher for each study time than the lowest dose of NGF.

Without wishing to bind to any interpretative theory, the Applicant believes that the unexpected tolerability of NGF 1-14 is due to the selectivity of action of the NGF 1-14 peptide towards the receptor tyrosine kinase TrKA with respect to the second receptor activated by NGF p75.

In conclusion, the NGF 1-14 peptide tested in vitro on primary cell cultures of normal human melanocytes results in a comparable increase in melanin content per cell compared to the whole protein even if at higher concentrations.

Surprisingly with respect to in vitro activity, the NGF 1-14 peptide tested in vivo on a mouse model of human vitiligo shows a more pronounced skin repigmentation, at the same dose, compared to the whole protein and a greater increase in the density of nerve endings, at the same dose.

Finally, the NGF 1-14 peptide administered intra-implant to Sprague-Dawley rats shows better tolerability than the NGF protein even at higher doses than those of the reference molecule. The result of the intra-implant test is very important, in light of the fact that the poor skin tolerability of NGF in intradermal tests is well known.

A further advantage of NGF1-14 over the whole protein is that, unlike the whole protein, it can be easily synthesized with conventional peptide synthesis approaches and stored under mild conditions and for prolonged periods, with a consequent significant reduction in production costs.

Therefore, the NGF 1-14 peptide such as peptides having sequence with at least 85%, preferably at least 90%, and preferably at least 95% identity with it and length up to 16 amino acids, are particularly useful in the treatment and/o in the prevention of skin dyschromia, such as vitiligo and skin de-innervation diseases.

Such oligo-peptide derivatives of NGF can therefore find application in the treatment and/or prevention of skin dyschromia, such as vitiligo, in a more effective and safe way than the cortisone derivatives currently used.

Furthermore, such oligo-peptide derivatives of NGF can find application in the treatment and/or prevention of skin de-innervation diseases, such as for example herpes, assisting antiviral therapy.

1. A method for the treatment and/or prevention of skin dyschromia and/or a de-innervation disease of the skin of a subject, the method comprising administering to the subject:
a peptide of 14 amino acids having the sequence ID No. 1,
a peptide of up to 16 amino acids having a sequence with at least 85%, preferably at least 90%, and preferably at least 95% identity with sequence ID No. 1, or
a derivative and/or salt thereof.

2. The method according to claim 1, wherein the peptide or derivative and/or salt thereof has a sequence with at least 85%, preferably at least 90%, and preferably at least 95% identity with the sequence ID No. 1, and has a length of up to 15 amino acids, more preferably of 14 amino acids.

3. The method according to claim 1, wherein said derivative or salt thereof includes an N and/or a C terminal of said peptide which is chemically modified or protected with an organic compound.

4. The method according to claim 3, wherein said compound is selected from the group consisting of phosphoryl, glycosyl, acyl, alkyl, carboxyl, hydroxyl, biotinyl, ubiquitinyl, and amide.

5. A method for the treatment and/or prevention of skin dyschromia and/or a de-innervation disease of the skin in a subject, the method comprising administering to the subject a pharmaceutical composition consisting of:
a peptide of 14 amino acids having the sequence ID No. 1,
a peptide of up to 16 amino acids having a sequence with at least 85%, preferably at least 90%, and preferably at least 95% identity with sequence ID No. 1, or
a derivative and/or salt thereof,
and at least one pharmaceutically acceptable excipient.

6. The method according to claim 5, wherein said composition comprises from 10 to 10,000 μg/mL, preferably from 100 to 5,000 μg/mL, and more preferably from 200 to 4,000 μg/mL of said peptide or said derivative and/or salt thereof.

7. The method according to claim 5 wherein the pharmaceutical composition is administered topically, preferably in the form of lotion, cream, gel, stick, spray, ointment, paste, mousse.

8. The method according to claim 5, wherein said at least one pharmaceutical acceptable excipient is selected from the group consisting of: pharmaceutically acceptable vehicles, volatile and non-volatile solvents, water, surfactants, preservatives, absorbents, chelators, lubricants, moisturisers, water repellents, antioxidants, UV absorbers, anti-irritants, vitamins, trace metals, antimicrobial agents, perfumes, dyes and colored ingredients, and/or structuring agents.

9. The method according to claim 1, wherein said skin dyschromia is selected from the group consisting of: vitiligo, bilateral vitiligo, acrofacial vitiligo, generalized vitiligo, focal vitiligo, segmental vitiligo, universal vitiligo, perinevic or Sutton's nevus vitiligo, leukoderma, skin dyschromia, piebaldism, pityriasis alba, pityriasis versicolor, idiopathic and post-inflammatory guttata hypomelanosis, acromic or depigmented nevi, progressive macular hypomelanosis, hypomelanosis caused by metabolic or nutritional or endocrine disorders, hypomelanosis caused by chemical, physical or pharmaceutical agents, infectious and post-infectious hypomelanosis, and inflammatory edipomelanosis.

10. The method according to claim 9, wherein the peptide or derivative and/or salt thereof is administered in combination with a steroid-based or calcitriol-based compound or composition.

11. The method according to claim 1, wherein said de-innervation disease of the skin is selected from the group consisting of: trigeminal trophic syndrome, post-herpes zoster itching, dysesthesia of the scalp, parestetic notalgia, brachioradial itching, parestetic meralgia, itching associated with keloids or burn scars, and neuropathy of small fibers in the course of systemic diseases.

12. The method according to claim 11, wherein the peptide or derivative and/or salt thereof is administered in combination with an antiviral compound or composition.


SEQUENCE LISTING (1 sequence)

SEQ ID NO: 1 - protein, 14 aa, Artificial Sequence
  Synthetic Peptide
SSSHPIFHRGEFSV